(19) United States
(12) Patent Application Publication
(10) Pub. No.: US 2020/0126677 A1
(43) Pub. Date: Apr. 23, 2020

(54) DIGITAL PATIENT ENGAGEMENT

(71) Applicant: Medtronic, Inc., Minneapolis, MN (US)

(72) Inventors: Matic Meglic, Lausanne (CH); Irénée Anthelme Denis Marie Colin, Lausanne (CH)

(21) Appl. No.: 16/168,424

(22) Filed: Oct. 23, 2018

(51) Int. Cl. G16H 80/00; G16H 40/63; G16H 10/60; G16H 40/67

(52) U.S. Cl. CPC G16H 80/00; G16H 40/63; A61B 5/0022; G16H 40/67; G16H 10/60

(57) ABSTRACT

Methods, systems, and devices for managing digital communications with a patient are described. The method may include identifying a content configuration for a patient portal based on a type of medical procedure scheduled for the patient. The method may also include receiving an electronic inquiry message from the patient via an electronic messaging application. The method may further include determining, automatically by the electronic messaging application, a response to the received electronic inquiry message based on content of the electronic inquiry message and recorded medical information associated with the patient. In some cases, the method may include updating the content configuration for the patient portal based at least in part on the content of the electronic inquiry message and the response.

### BACKGROUND

The following relates generally to managing digital communications with a patient, and more specifically to digital patient engagement.

In a healthcare facility such as a hospital, physiological parameters of the patient (e.g., heart rate, respiratory rate, blood pressure) may be monitored by one or more medical devices. The medical devices may he battery powered and may wirelessly transmit measured patient data over a wireless network within the hospital, thereby allowing the patient to move freely through the hospital while being monitored. Clinicians may remotely monitor the patient by accessing the patient data at a central nurse station or on any web enabled device connected to the network (e.g., smartphone or tablet).

Before the patient enters the healthcare facility or after the patient returns home from the healthcare facility, the patient may ask questions or receive directions to perform follow-up consultations via the patient's personal device. However, the patient may be unable to communicate with a system designed to aggregate information associated with the patient or support the patient before, during, and after a medical procedure. In that case, the physical and physiological state of the patient may be at risk, and the patient recovery time may increase following the medical procedure

### SUMMARY

The described features generally relate to methods, systems, devices, or apparatuses that support digital patient engagement. A patient management server may identify a content configuration for a patient portal based on a type of medical procedure scheduled for the patient and other data (e.g. patient demographics, modifiable risk factors, digital literacy and/or patient access to digital devices and the internet, etc.) The patient management server may also receive an electronic inquiry message from the patient. For example, the patient may ask a question regarding preparation for the medical procedure or a recovery regime for the medical procedure via an electronic messaging application.

Based on the content of the inquiry message, the medical information stored and associated with the patient, and/or a trigger condition (e.g., time to the medical procedure, patient reported information, or vital sign measurement), the patient management server may determine a response to the inquiry message or other action. For example, the response may include a nutritional recommendation, an exercise recommendation, a video image, a connection to a clinical support center, a connection to a care team member, or a combination thereof. In some cases, the patient management server may update the content configuration for the patient portal based on the content of the inquiry message and the response. For example, the content configuration for the patient portal may be tailored on an individual patient basis according the type of medical procedure, the demographics of the patient, or a physiological value associated with the medical condition of the patient. That is, the patient care instructions may be configured and displayed according to patient specific characteristics.

A method for managing digital communications with a patient is described. The method may include identifying a content configuration for a patient portal based at least in part on a type of medical procedure scheduled for the patient, receiving an electronic inquiry message via an electronic messaging application from the patient, determining, automatically by the electronic messaging application, a response to the received electronic inquiry message based at least in part on content of the electronic inquiry message and recorded medical information associated with the patient, and updating the content configuration for the patient portal based at least in part on the content of the electronic inquiry message and the response.

In some examples of the method described herein may further include determining patient care instructions based at least in part on the content of the electronic inquiry message and the recorded medical information associated with the patient and transmitting, to the patient, the determined patient care instructions. In some examples of the method described herein may further include displaying, via a graphical user interface, the content configuration for the patient portal, wherein the content configuration comprises the determined patient care instructions.

In some examples of the method described herein may further include determining an intervention action to respond to the electronic inquiry message based at least in part on the content of the electronic inquiry message and the recorded medical information associated with the patient and transmitting, to a clinician, a first request for the intervention action based at least in part on determining the intervention action. In sonic examples of the method described herein may further include determining an intervention action to respond to the electronic inquiry message based at least in part on the content of the electronic inquiry message and the recorded medical information associated with the patient and transmitting, to a patient support system, a first request for the intervention action based at least in part on determining the intervention action. In same examples of the method described herein may further include receiving an indication of the intervention action for displaying in the content configuration, wherein the indication of the intervention action is received from the patient support system, the clinician, or both.

In some examples of the method described herein may further include updating the recorded medical information associated with the patient in a database of a patient management system based at least in part on determining the response. In some examples of the method described herein may ⁻further include transmitting, to a patient management system, a patient communication history record associated with communication between the patient and the electronic messaging application. In some examples of the method described herein may further include transmitting, to a patient management system, an electronic status indicator associated with a status of the patient after a completion of the type of medical procedure scheduled for the patient. In some examples of the method described herein may further include transmitting, to the patient, an electronic reminder message associated with the response to the received electronic inquiry message, a schedule of the type of medical procedure, or both and receiving, from the patient, an electronic confirmation message indicating that the patient received the electronic reminder message, completed a task associated with the electronic reminder message, or both.

In some examples, the content configuration may comprise a recommendation to prepare for the type of medical procedure scheduled for the patient, a recovery process recommendation for the type of medical procedure scheduled for the patient, a nutritional recommendation, an exercise recommendation, a video image associated with a recommendation, a graphical image associated with a recommendation, a textual image associated with a recommendation, or a combination thereof. In some examples, the recorded medical information associated with the patient may comprise a medical condition associated with the patient, a demographic characteristic associated with the patient, a physiological measurement value, a patient generated response, a date of the type of medical procedure, or a combination thereof.

Some examples of the method described herein may further include operations, features, means, or instructions for identifying a content configuration for a patient portal based at least in part on a type of medical procedure scheduled for the patient, receiving an electronic inquiry message via an electronic messaging application from the patient, determining, automatically by the electronic messaging application, a response to the received electronic inquiry message based at least in part on content of the electronic inquiry message and recorded medical information associated with the patient, and updating the content configuration for the patient portal based at least in part on the content of the electronic inquiry message and the response.

### BRIEF DESCRIPTION OF THE DRAWINGS

FIG. 1 illustrates an example of a system for managing digital communications with a patient that supports digital patient engagement in accordance with aspects of the present disclosure.

FIG. 2 illustrates an example of a system that supports digital patient engagement in accordance with aspects of the present disclosure.

FIG. 3 illustrates an example of a patient engagement system that supports digital patient engagement in accordance with aspects of the present disclosure.

FIG. 4 illustrates an example of a process flow that supports digital patient engagement in accordance with aspects of the present disclosure.

FIGS. 5 and 6 show block diagrams of devices that support digital patient engagement in accordance with aspects of the present disclosure.

FIG. 7 shows a block diagram of a communications manager that supports digital patient engagement in accordance with aspects of the present disclosure.

FIG. 8 shows a diagram of a system including a device that supports digital patient engagement in accordance with aspects of the present disclosure.

FIGS. 9-11 show a flowchart illustrating methods that support digital patient engagement in accordance with aspects of the present disclosure.

### DETAILED DESCRIPTION

In some patient care systems, a patient may return home from the healthcare facility with instructions for at-home patient care after a medical procedure or appointment. In some cases, the patient may be instructed to follow a set of recommendations to prepare for the medical procedure or the appointment. In that case, the patient may interact with a patient engagement system. For example, the patient engagement system may be tailored on a patient by patient basis according to the patient's medical condition, characteristics, a physiological measurement value, or a combination thereof.

In some cases, the patient may interact with the patient engagement system via a digital platform accessible from web-enabled device such as a phone, tablet, or computer. The patient engagement system may include an application configured to interact with the patient and receive electronic inquiry messages. In some cases, the application may provide a means of communication between the patient and an online coach via one or more messages. In other examples, the patient engagement system may allow the patient to interact with a patient support center that may instruct the patient how to manage stress, eat a well-balanced diet, or implement an exercise program into the patient's routine. In that case, the patient engagement system may provide the patient with recommendations to prepare for the type of medical procedure, a recovery process recommendation for the type of medical procedure, or both.

Based on the content of the electronic inquiry message or other trigger condition, the patient engagement system may include a patient management server that determines patient care instructions for the patient. In some cases, the content of the electronic inquiry message may determine that an intervention may be required from a clinician or patient support center. In that case, the first line responsible actor (e.g., clinician or patient support center) ay receive a request to respond to the electronic inquiry message. If the first line responsible actor does not respond or receive the request, then the request may be transferred to the second line responsible actor. The second line responsible actor may receive the request and transmit a response to the patient via the patient engagement system.

The patient engagement system may also communicate bidirectionally with the patient. For example, the patient management server may transmit reminder messages to the patient indicating an uncompleted task and receive a confirmation message from the patient indicating a completed task. In sonic cases, the patient management server may document communication history between the patient and the application and store the communication history in a database associated with the patient engagement system. In other examples, the patient may transmit a status indicator to the patient engagement system to indicate a status of the patient after the completion of the medical procedure.

The patient engagement system may provide a platform to support the patient before, during, and after the medical procedure. For example, the patient engagement system may reduce the patient's anxiety before the medical procedure in addition to allowing the patient access to a support channel to communicate with the clinician or support center. In that case, the patient's physical, nutritional, and psychological state may improve and allow for a faster recovery time following the medical procedure,

Aspects of the disclosure are initially described in the context of a wireless patient monitoring system. Aspects of the disclosure are further illustrated by and described with reference to apparatus diagrams, system diagrams, and flowcharts that relate to digital patient engagement.

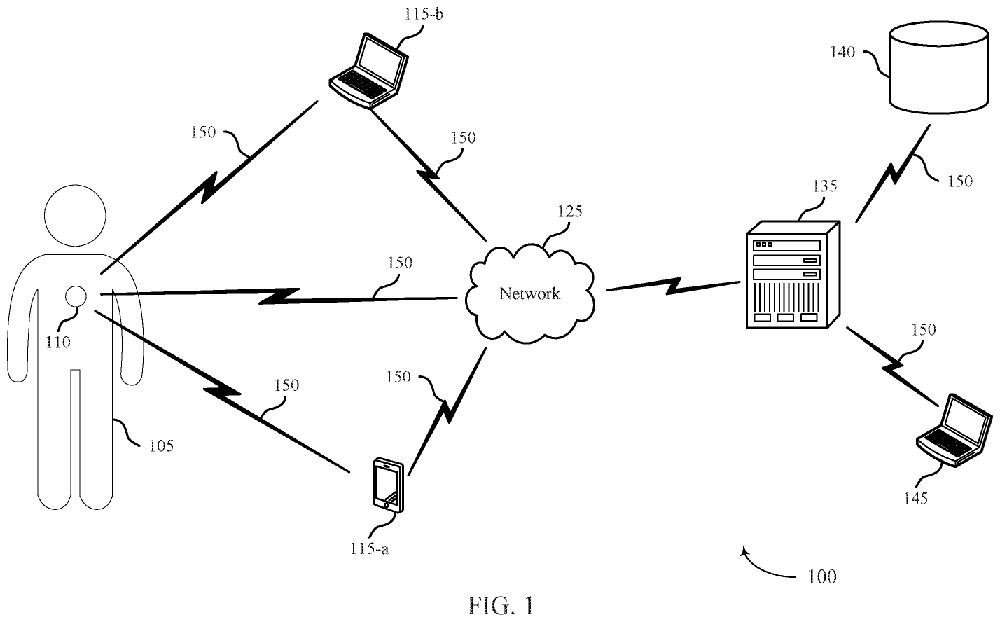

FIG. 1 illustrates an example of a system 100 in accordance with various embodiments of the present disclosure. The system 100 may include a patient 105 wearing, carrying, or otherwise coupled with a medical device 110. Although a single medical device 110 is shown, multiple medical devices 110 may be coupled to the patient 105. The patient 105 may be a patient at home, in a hospital, nursing home, home care, a medical facility, or another care facility. The medical device 110 may transmit signals via wireless communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105. In some cases, the medical device 110 associated with the patient 105 may communicate the variety of physiological parameters to a patient management system in order to configure the content configuration for a patient portal.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.

Computing device 115-a may be a wireless device such as a tablet, cellular phone, personal digital assistant (PDA), a dedicated receiver, or other similar device or a spatially distributed network of devices configured to receive signals from the medical device 110. Computing device 115-b may be a wireless laptop computer configured to receive signals from the medical device 110. The computing devices 115 may be in communication with a central station 135 via network 125.

The medical device 110 may also communicate directly with the central station 135 via the network 125. The central station 135 may be a server located within the hospital or in a remote location. The central station 135 may be in further communication with one or more remote computing devices 145, thereby allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

In accordance with various embodiments, methods and apparatuses are described for digital patient engagement. As a patient 105 prepares for a medical procedure (e.g., a surgery) or after the patient 105 returns home after an appointment with the clinician or a medical procedure, patient 105 may be monitored based on a medical procedure, or patient 105 may have follow-up questions based on their medical procedure. In that case, the patient may have access to a patient portal to ask questions, receive recommendations for patient care, or both. For example, a patient management server (e.g., central station 135) may identify a content configuration for the patient portal based on a type of medical procedure scheduled for the patient.

The patient management server may also receive, from the patient 105, an electronic inquiry message via an electronic messaging application. The patient management server may determine, automatically by the electronic messaging application, a response to the received inquiry message based on content of the message and recorded medical information associated with the patient 105. In some cases, the patient management server may update the content configuration for the patient portal based on the content of the message and the response.

In some examples, the content configuration of the patient portal may be customized according to various parameters. For example, the content configuration may be customized according to a medical condition associated with the patient 105, a demographic characteristic associated with the patient 105 (e.g., age, gender), a physiological measurement value, or a combination thereof. In that case, the content configuration may include specific details on a per patient, per medical procedure basis. That is, the content configuration may include the characteristics associated with the patient 105.

In conventional systems, a patient engagement system may be configured the same for multiple patients 105. That is, a conventional patient engagement system may support a single medical condition or medical procedure for multiple patients 105. In some cases, the content displayed to the patient 105 may omit changes or adjustments to the preferences or characteristics of the patient 105. In that case, the content displayed to the patient 105 may be static. For example, the content displayed may be updated according to a change in the medical procedure rather than a communication history between the patient 105 and the conventional patient engagement system.

In accordance with aspects of the present disclosure, to efficiently handle more than one patient 105 as well as more than one medical procedure, the patient engagement system may be multi-indication for multiple patients 105. In that case, the patient engagement system may include a single platform for more than one medical procedure (e.g., multi-indication) and more than one patient 105. In some cases, the patient engagement system may communicate with an external system (e.g., including a clinician or support member) for assisting in an intervention action if indicated in the message received from the patient 105. In some cases, the content configuration of the patient portal may be dynamically updated according to a communication between the patient 105 and the patient engagements system. For example, each interaction between the patient 105 and the patient engagement system may be stored within the patient engagement system and automatically update the content configuration.

To securely handle a multi-indication and multi-tenant patient engagement system, the patient engagement system may include a level of security and encryption. For example, the electronic messaging application may be encrypted. In some cases, the identification name of the patient 105 may be encrypted prior to indexing the patient's information or recorded medical information in an associated database 140. In that case, the index of the database 140 may be encrypted to ensure a level of security when the patient management server updates the recorded medical information associated with the patient 105 in the database 140 of the patient management system.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

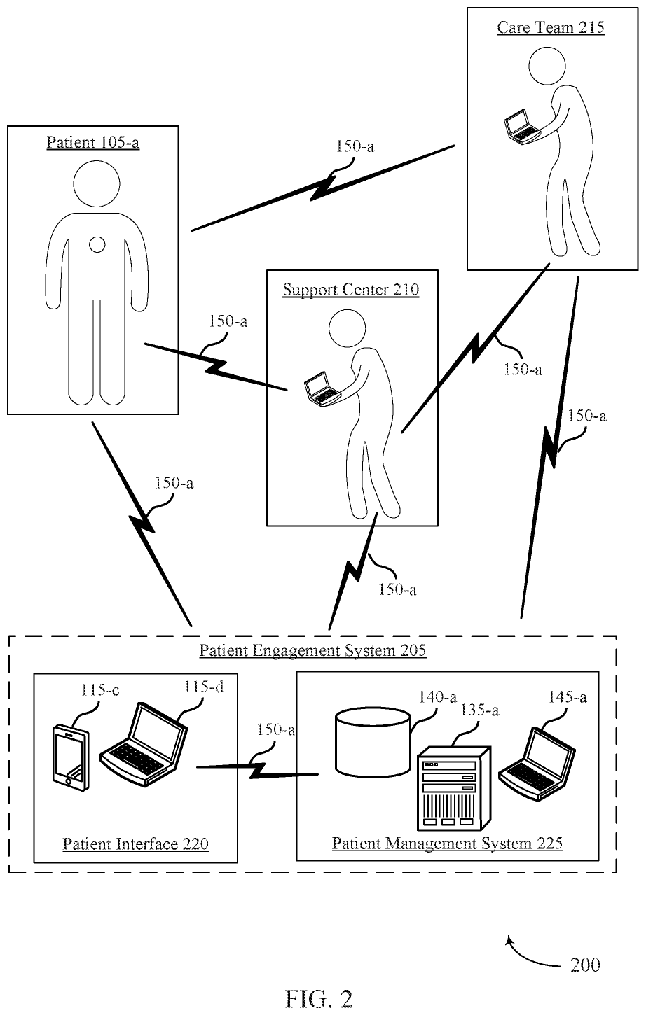

FIG. 2 illustrates an example of a system 200 that supports digital patient engagement in accordance with aspects of the present disclosure. In some examples, system 200 may implement aspects of system 100. System 200 may include patient 105-a, patient engagement system 205, support center 210, and care team 215.

100391 Patient engagement system 205 may include patient interface 220 and patient management system 225. Patient interface 220 may communicate bidirectionally via wired or wireless communication links 150-a to patient management system 225. Patient interface 220 may include computing devices 115-c and 115-d which may be a tablet, cellular phone, or computer configured to display the content configuration of the patient portal. In some cases, computing devices 115-c and 115-d may interface between patient 105-a and patient management system 225. Computing devices 115-c and 115-d may be examples of computing device 115 as described with reference to FIG. 1.

Patient management system 225 may include database 140-a, patient management server 135-a, and remote computing device 145-a, which may be respective examples of database 140, central station 135, and remote computing device 145 as described with reference to FIG. 1. In some cases, care team 215 and support center 210 may interface with the patient portal via the remote computing device 145-a.

In some cases, patient management server 135-a may identify the content configuration for the patient portal associated with patient 105-a. For example, the content configuration may be identified based on the type of medical procedure associated with patient 105-a. Patient 105-a may send a message (e.g., electronic inquiry message) to patient engagement system 205 via communication link 150-a. For example, patient 105-a may interact with an application (e.g., electronic messaging application) via computing device 115-c or 115-d to send the message. The message may include a question regarding the type of medical procedure, how to prepare for the medical procedure, specifics regarding the date, place, and time of the medical procedure, expectations for the recovery process after the medical procedure, or a combination thereof.

Patient management server 135-a may determine, automatically by the application, a response to the message. For example, the response may be based on the content of the message and recorded medical information associated with patient 105-a. In some cases, patient management server 135-a may be in electronic communication with database 140-a and may access recorded medical information stored in database 140-a. For example, the recorded medical information may include a medical condition associated with the patient, a demographic characteristic associated with the patient, a physiological measurement value, or a combination thereof.

Patient engagement system 205 may also collect data from the patient 105-a via a questionnaire to update the recorded medical record stored in database 140-a. For example, database 140-a may comprise of a customer relationship management (CRM) database. In some cases, database 140-a may also store a library of content configurations that may be utilized across different medical procedures for patients 105-a.

Based on the content of the message and the response, the content configuration for the patient portal may be updated and accessible for patient 105-a to view the content configuration via patient interface 220. For example, patient management server 135-a may determine patient care instructions according to the content of the message and display the patient care instructions to patient 105-a via a graphical interface. Computing device 115-c or 115-d may include the graphical interface.

In some cases, the content of the message may indicate that an intervention is required from a clinician (e.g., care team 215) according to an escalation protocol associated with patient 105-a. For example, patient 105-a may send a message to patient engagement system 205 indicating that the message may he transferred to care team 215. The message may include a request for confidential patient specific information, a request regarding an escalated medical condition associated with the medical procedure, or both. In that case, care team 215 may receive the message and respond to patient 105-a via communication link 150-a. For example, care team 215 may directly communicate with patient 105-a, or care team 215 may transmit the response to patient engagement system 205. Care team 215 may also access the recorded medical information associated with patient 105-a via remote computing device 145-a.

In some cases, care team 215 or support center 210 may be unresponsive or unable to respond to the message. In that case, the message may be transmitted to the other actor e.g., support center 210 or care team 215). Support center 210 may receive the message and respond to patient 105-a via communication link 150-a. For example, support center 210 may directly communicate with patient 105-a, or support center 210 may transmit the response to patient engagement system 205. In some cases, the initial message may indicate that the message may he transferred to support center 210. For example, the message may include a request for troubleshooting a problem with the medical device associated with the medical procedure, a request regarding patient engagement system 205 (e.g., patient interface 220), or both. In some examples, support center 210 may provide information on various aspects of the medical procedure. In other examples, support center 210 may provide advice on technical aspects regarding the electronic messaging application. In some cases and depending on the content configuration, if a lack of response from support center 210 is detected, the message may be transmitted to care team 215 In other examples, if a lack of response from care team 215 is detected, the message may be transmitted to support center 210.

In some cases, care team 215 or support center 210 may interact with the patient engagement system 205 to differentiate between individual patients 105-a and improve the patient's fitness for the medical procedure. In some cases, care team 215 may utilize the patient engagement system 205 to collect perioperative data associated with the medical procedure and improve the quality of care and experience for the patient 105-a.

The patient management server 135-a may receive the response from support center 210 and care team 215, and may display the response in the content configuration. As described below in more detail, the content configuration may include a recommendation to prepare for the type of medical procedure scheduled for the patient 105-a, a recovery process recommendation for the type of medical procedure, a nutritional recommendation, an exercise recommendation, a video image associated with a recommendation, a graphical image associated with a recommendation, a textual image associated with a recommendation, or a combination thereof.

In some cases, an informal caregiver (e.g., a spouse, child, parent, friend, etc.) may interact with the patient engagement system 205. For example, the patient 105-a may be unable to interact with the patient engagement system 205. In such cases, the informal caregiver may utilize the patient engagement system 205 to seek assistance from care team 215 or support center 210. In some cases, the informal caregiver may request patient care instructions associated with the patient 105-a.

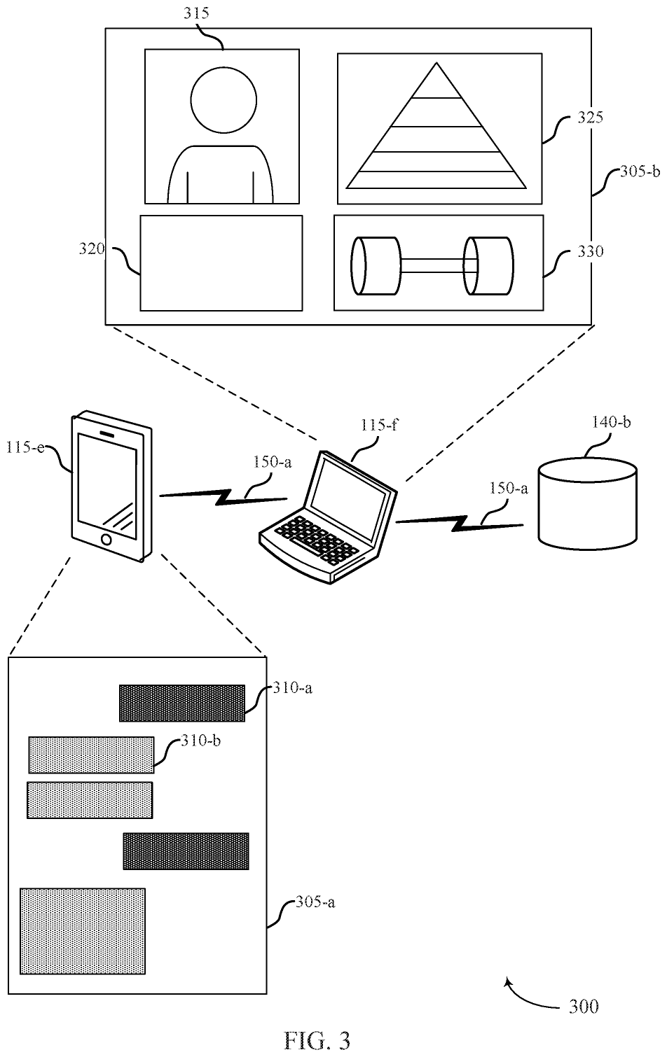

FIG. 3 illustrates an example of a patient engagement system 300 that supports digital patient engagement in accordance with aspects of the present disclosure. In some examples, patient engagement system 300 may implement aspects of 100 and system 200. Patient engagement system 300 may include computing device 115-e, computing device 115-f, and database 140-b which may he respective examples of computing device 115 and database 140 as described with reference to FIGS. 1 and 2.

In some cases, computing device 115-e may include a graphical user interface 305-a for displaying a messaging application. For example, graphical user interface 305-a may include message 310-a and message 310-b. Message 310-a may be an example of an electronic inquiry message, message 310-b may be an example of an electronic response. In some cases, the patient may send message 310-a via a messaging application, and computing device 115-e may display message 310-a via graphical user interface 305-a. For example, the messaging application may be an example of an artificial intelligence driven chatbot. The messaging application may have been previously trained (e.g., using supervised learning) on labeled pairs of electronic messages 310-a from a patient and correct responses (e.g., messages 310-b). Therefore, in response to a message 310-a, the chatbot may classify the message 310-a and serve up (e.g., determine) a response 310-b that corresponds to the message 310-a. Message 310-a may include an inquiry regarding the type of medical procedure, preparation for the medical procedure, the date, place, and time of the medical procedure, expectations after completing the medical procedure, or a combination thereof.

The messaging application may determine message 310-b (e.g., response) to message 310-a and display message 310-b via graphical user interface 305-a. Message 310-b may include an answer to the inquiry included in the message 310-a, a transfer to an alternative support system, or both. For example, message 310-b may be based on a response from a care team (e.g., clinician), a support center, or both. In some cases, message 310-a and message 310-b may comprise a patient communication history. In that case, computing device 115-e may transmit the patient communication history via communication links 150-a to database 140-b (e.g., patient management system). In some cases, the patient communication history may be embedded into an application (e.g., web-based application) of computing device 115-f.

In some cases, computing device 115-e may transmit a status indicator request to the patient via message 310-a. For example, computing device 115-e may request a status of the patient after the patient completed the type of medical procedure. In that case, the patient may transmit their status after completing the medical procedure via message 310-b. The status may include a physiological measurement value associated with the medical procedure or an indicator of the patient's physiological, mental or emotional condition.

In other examples, computing device 115-e may transmit a reminder message to the patient via message 310-a, For example, the reminder message may include a reminder for the patient to exercise, take a medication associated with the medical procedure, eat a well-balanced diet, consult a clinician or support center if necessary, or a combination thereof. In that case, the patient may transmit a confirmation message via message 310-b to computing device 115-e. The confirmation message may include a message indicating that the patient received the reminder message, completed a task associated with the reminder message, or both.

In some cases, the patient may log into a web application via computing device 115-f. Computing device 115-f may display the content configuration of the patient portal via graphical user interface 305-b. In sonic cases, computing device 115-e may display the content configuration of the patient portal via graphical user interface 305-a. The interaction between the patient and the application in computing device 115-e may also be applicable to the interaction between the patient and the web-based application within computing device 115-f. For example, the web-based application within computing device 115-f may also send reminder messages, send the status indicator request, and receive messages from the patient.

Graphical user interface 305-b may configure the content configuration according to the recorded medical information associated with the patient in database 140-b as well as the content of the message and response. For example, graphical user interface 305-b may display patient care instructions in response to receiving the message. The patient care instructions may include a recommendation to prepare for the type of medical procedure scheduled for the patient, a recovery process recommendation for the type of medical procedure scheduled for the patient, or both via a video feed 315. For example, video feed 315 may include a video to prepare the patient for the medical procedure by providing instructions for packing for the hospital stay, information regarding the hospital stay, and directions to the hospital. In some cases, the patient care instructions may include a personalized message or reminder. The personalized message or reminder may be based on patient demographics, the type of medical procedure, a date of the type of medical procedure, a risk factor associated with the type of medical procedure, or a combination thereof.

In some cases, the patient care instructions may include a graphical image associated with a recommendation such as a nutritional recommendation 325, an exercise recommendation 330, or both. In some examples, the patient care instructions may include a textual image 320 associated with a recommendation. For example, the textual image may include instructions regarding the nutritional recommendation 325, the exercise recommendation 330, or bath.

In some cases, patient engagement system 300 may include medical procedure specific engagement mechanisms for each individual patient. For example, graphical user interface 305-a and 305-b may display content configuration that may be tailored to the patient. For example, the content configuration for a patient with chronic obstructive pulmonary disease (COPD) may be different than for a patient with a bone fracture. That is, exercise recommendation 330 for the patient with COPD may be different than exercise recommendation 330 for the patient with the bone fracture. In some cases, the content configuration may be displayed according to an age of the patient. For example, an adolescent patient may interact with the patient portal and content configuration differently than an elderly patient. That is, the content configuration may be updated and displayed on a patient by patient basis.

Patient engagement system 300 may dynamically update the content configuration according to an interaction between the patient and the patient engagement system 300. For example, the patient may receive a customized care-plan via user interface 305-b. That is, the care-plan may include nutritional recommendation 325 according to the age, gender, weight, medical condition, or other stored information associated with the patient. The nutritional recommendation 325 may display the recommendation in the form of a chart, graph, or table illustrating the suggested types of food and amount of food.

The patient 105 may send message 310-a including an inquiry on what time of day to eat the food. In some cases, the database 140-b may store an indication associated with the content of the inquiry message, and the patient management system 300 may dynamically update the content displayed via user interface 305-b. For example, the patient management system 300 may determine the time of day to eat the food and respond to the patient via message 310-b. For example, message 310-b may include a link to download and access a suggested meal plan.

Message 310-b may also include a reminder message for the patient to eat at the suggested time of day. When the patient completes the task, the patient may send a confirmation message via message 310-a. The patient management system 300 may then transmit an indication of the confirmation to a member of the care team (e.g., dietician) indicating that the patient completed nutritional recommendation 325. In other examples, the patient management system 300 may update an overall adherence or specific module adherence indicator associated with the completed task. In some cases, the patient management system 300 may determine that the patient completed nutritional recommendation 325 and may update the content configuration according to a second nutritional recommendation 325.

In some cases, the content configuration may include a quiz or knowledge test for patients to test their understanding of the patient care instructions. In some cases, the result of the quiz or knowledge test may be used to determine a module or activity associated with the patient care instructions. In some examples, the content configuration may include an interactive experience where the patient may interact with the content configuration. The patient may be awarded points or medals based on the results of the interactive experience. For example, the patient may receive a point or virtual medal that may be associated with the completion of a task or a score of the quiz or knowledge test.

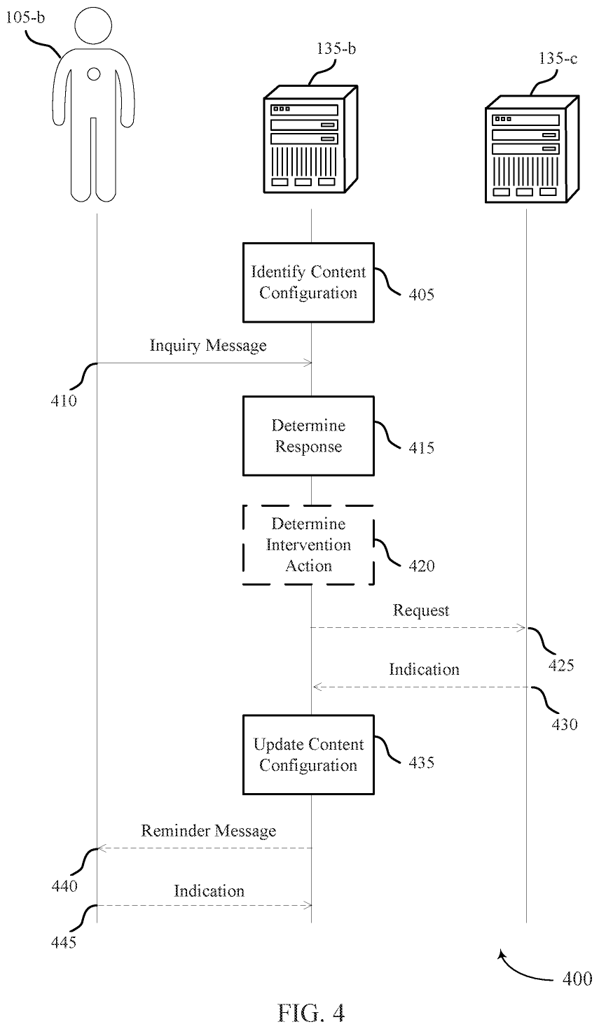

FIG. 4 illustrates an example of a process flow 400 that supports digital patient engagement in accordance with aspects of the present disclosure. Process flow 400 may include patient 105-b, patient management server 135-b, and patient support server 135-c, which may be respective examples of patient 105 and central station 135 as described with reference to FIGS. 1-3. The patient support server 135-c may also be an example of a patient management support system as described with reference to FIGS. 1 and 2. Alternative examples of the following may be implemented, where some steps are performed in a different order or not at all. Some steps may additionally include additional features not mentioned above.

At block 405, patient management server 135-b may identify content configuration. Patient management server 135-b may receive inquiry message 410. At block 415, patient management server 135-b may determine a response. For example, patient management server 135-b may determine, automatically by the electronic messaging application, a response to the received electronic inquiry message based at least in part on content of the electronic inquiry message and recorded medical information associated with the patient. In some cases, patient management server 135-b may determine patient care instructions based at least in part on the content of the electronic inquiry message and the recorded medical information associated with the patient and trans it, to the patient, the determined patient care instructions.

In some cases, the content configuration may comprise a recommendation to prepare for the type of medical procedure scheduled for the patient, a recovery process recommendation for the type of medical procedure scheduled for the patient, a nutritional recommendation, an exercise recommendation, a video image associated with a recommendation, a graphical image associated with a recommendation, a textual image associated with a recommendation, or a combination thereof. In other examples, the recorded medical information associated with the patient may comprise a medical condition associated with the patient, a demographic characteristic associated with the patient, a physiological measurement value, a patient generated response (e.g., via a questionnaire), or a combination thereof.

At block 420, patient management server 135-b may determine an intervention action to respond to the electronic inquiry message based at least in part on the content of the electronic inquiry message and the recorded medical information associated with the patient. In some cases, patient management server 135-b may determine an intervention action to respond to the electronic inquiry message based at least in part on the content of the electronic inquiry message and the recorded medical information associated with the patient. In other examples, patient management server 135-b may determine a lack of response from the patient support system to the first request for the intervention action.

Patient management server 135-b may transmit request 425. For example, patient management server 135-b may transmit, to a patient support system, a first request for the intervention action based at least in part on determining the intervention action. In some cases, patient management server 135-b may transmit, to a clinician, a second request for the intervention action based at least in part on determining the lack of response to the first request for the intervention action. In other examples, patient management server 135-b may transmit, to a clinician, a second request for the intervention action based at least in part on determining the lack of response to the first request for the intervention action.

Patient management server 135-b may receive indication 430. For example, patient management server 135-b may receive an indication of the intervention action for displaying in the content configuration, wherein the indication of the intervention action is received from the patient support server 135-c (e.g., the patient support system, the clinician, or both).

At block 435, patient management server 135-b may update content configuration. For example, patient management server 135-b may update the content configuration for the patient portal based at least in part on the content of the electronic inquiry message and the response. In some examples, patient management server 135-b may display, via a graphical user interface, the content configuration for the patient portal, wherein the content configuration comprises the determined patient care instructions.

In some cases, patient management server 135-b may update the recorded medical information associated with the patient in a database of a patient management system based at least in part on determining the response. In some examples, patient management server 135-b may transmit, to a patient management system, a patient communication history record associated with communication between the patient and the electronic messaging application. In other examples, patient management server 135-b may transmit, to a patient management system, an electronic status indicator associated with a status of the patient after a completion of the type of medical procedure scheduled for the patient.

Patient management server 135-b may transmit reminder message 440. For example, patient management server 135-b may transmit, to the patient 105-b, an electronic reminder message associated with the response to the received electronic inquiry message, a schedule of the type of medical procedure, or both. Patient 105-b may transmit indication 445. For example, patient management server 135-b may receive, from the patient 105-b, an electronic confirmation message (e.g., indication 445) indicating that the patient received the electronic reminder message, completed a task associated with the electronic reminder message, or both.

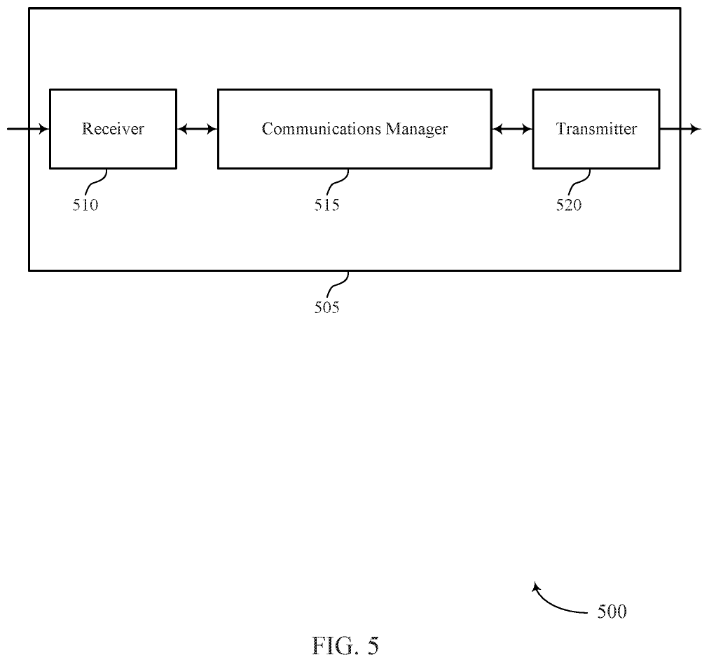

FIG. 5 shows a block diagram 500 of a patient management server 505 that supports digital patient engagement in accordance with aspects of the present disclosure. The patient management server 505 may be an example of aspects of a server as described herein. The patient management server 505 may include a receiver 510, a communications manager 515, and a transmitter 520. The patient management server 505 may also include a processor. Each of these components may be in communication with one another (e.g., via one or more buses).

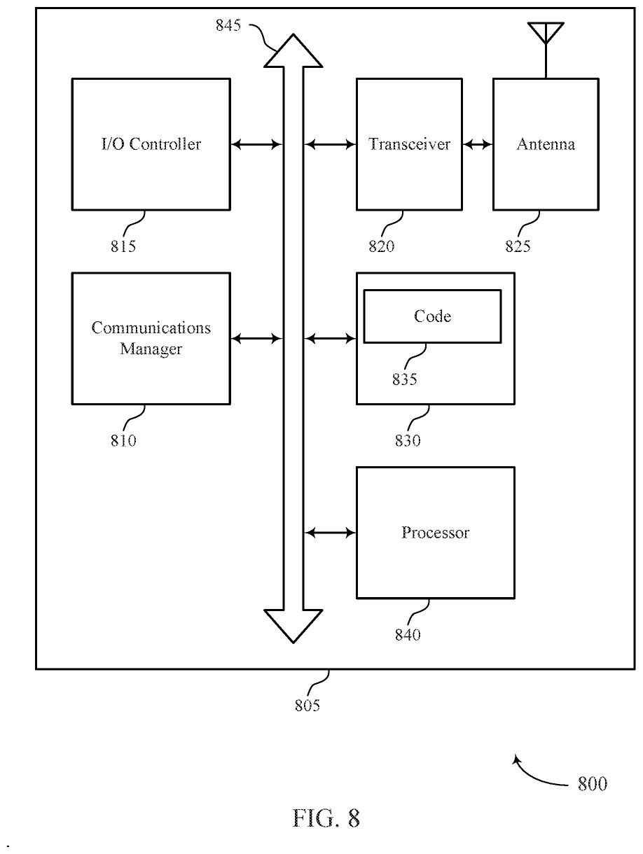

The receiver 510 may receive information such as packets, user data, or control information associated with various information channels (e.g., control channels, data channels, and information related to digital patient engagement, etc.). Information may be passed on to other components of the patient management server 505. The receiver 510 may be an example of aspects of the transceiver 820 described with reference to FIG. 8. The receiver 510 may utilize a single antenna or a set of antennas.

The communications manager 515 may identify a content configuration for a patient portal based on a type of medical procedure scheduled for the patient, receive an electronic inquiry message via an electronic messaging application from the patient, determine, automatically by the electronic messaging application, a response to the received electronic inquiry message based on content of the electronic inquiry message and recorded medical information associated with the patient, and update the content configuration for the patient portal based on the content of the electronic inquiry message and the response. The communications manager 515 may be an example of aspects of the communications manager 810 described herein.

The communications manager 515, or its sub-components, may be implemented in hardware, code (e.g., software or firmware) executed by a processor, or any combination thereof. If implemented in code executed by a processor, the functions of the communications manager 515, or its sub-components may be executed by a general-purpose processor, a DSP, an application-specific integrated circuit (ASIC), a FPGA or other programmable logic device, discrete gate or transistor logic, discrete hardware components, or any combination thereof designed to perform the functions described in the present disclosure.

The communications manager 515, or its sub-components, may be physically located at various positions, including being distributed such that portions of functions are implemented at different physical locations by one or more physical components. In sonic examples, the communications manager 515, or its sub-components, may be a separate and distinct component in accordance with various aspects of the present disclosure. In some examples, the communications manager 515, or its sub-components, may be combined with one or more other hardware components, including but not limited to an input/output (I/O) component, a transceiver, a network server, another computing device, one or more other components described in the present disclosure, or a combination thereof in accordance with various aspects of the present disclosure.

The transmitter 520 may transmit signals generated by other components of the patient management server 505. In some examples, the transmitter 520 may be collocated with a receiver 510 in a transceiver module. For example, the transmitter 520 may be an example of aspects of the transceiver 820 described with reference to FIG. 8. The transmitter 520 may utilize a single antenna or a set of antennas.

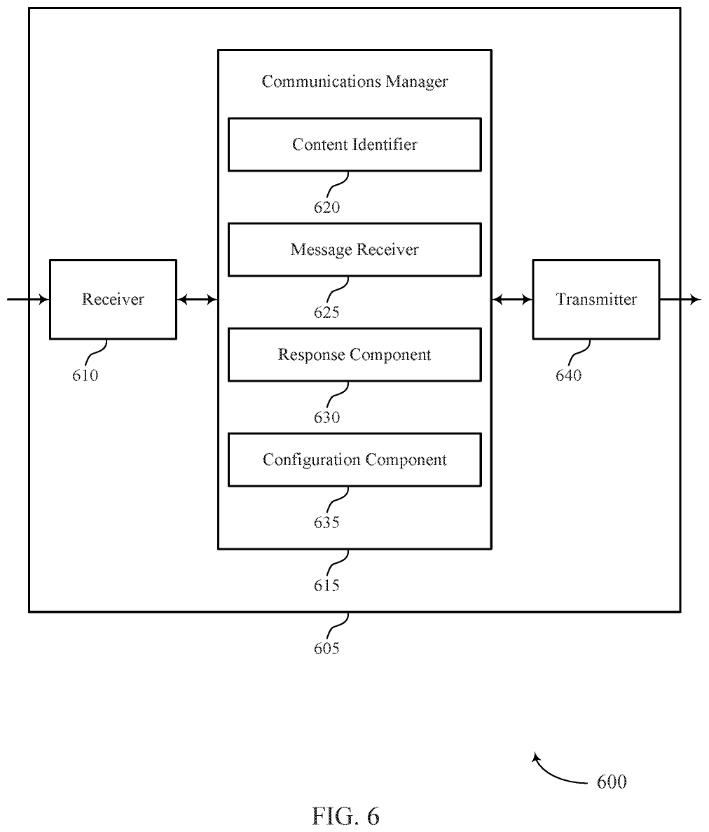

FIG. 6 shows a block diagram 600 of a patient management server 605 that supports digital patient engagement in accordance with aspects of the present disclosure. The patient management server 605 may be an example of aspects of a patient management server 505 as described herein. The patient management server 605 may include a receiver 610, a communications manager 615, and a transmitter 640. The patient management server 605 may also include a processor. Each of these components may be in communication with one another (e.g., via one or more buses).

The receiver 610 may receive information such as packets, user data, or control information associated with various information channels (e.g., control channels, data channels, and information related to digital patient engagement, etc.). Information may be passed on to other components of the patient management server 605. The receiver 610 may be an example of aspects of the transceiver 820 described with reference to FIG. 8. The receiver 610 may utilize a single antenna or a set of antennas.

The communications manager 615 may be an example of aspects of the communications manager 515 as described herein. The communications manager 615 may include a content identifier 620, a message receiver 625, a response component 630, and a configuration component 635. The communications manager 615 may be an example of aspects of the communications manager 810 described herein.

The content identifier 620 may identify a content configuration for a patient portal based on a type of medical procedure scheduled for the patient.

The message receiver 625 may receive an electronic inquiry message via an electronic messaging application from the patient.

The response component 630 may determine, automatically by the electronic messaging application, a response to the received electronic inquiry message based on content of the electronic inquiry message and recorded medical information associated with the patient.

The configuration component 635 may update the content configuration for the patient portal based on the content of the electronic inquiry message and the response.

The transmitter 640 may transmit signals generated by other components of the patient management server 605. In some examples, the transmitter 640 may be collocated with a receiver 610 in a transceiver module. For example, the transmitter 640 may be an example of aspects of the transceiver 820 described with reference to FIG. 8. The transmitter 640 may utilize a single antenna or a set of antennas.

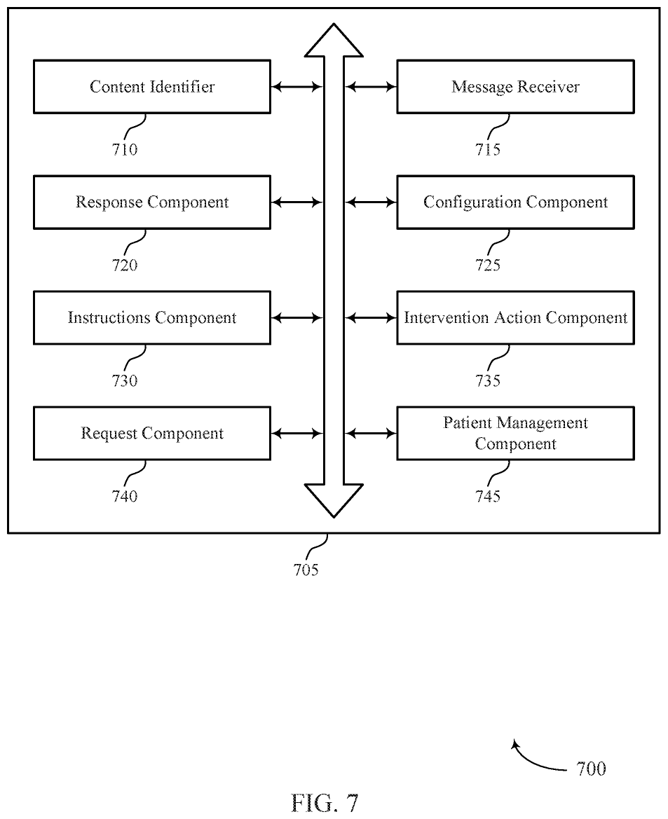

FIG. 7 shows a block diagram 700 of a communications manager 705 that supports digital patient engagement in accordance with aspects of the present disclosure. The communications manager 705 may be an example of aspects of a communications manager 515, a communications manager 615, or a communications manager 810 described herein.

The communications manager 705 may include a content identifier 710, a message receiver 715, a response component 720, a configuration component 725, an instructions component 730, an intervention action component 735, a request component 740, and a patient management component 745. Each of these modules may communicate, directly or indirectly, with one another (e.g., via one or more buses).

The content identifier 710 may identify a content configuration for a patient portal based on a type of medical procedure scheduled for the patient. In some cases, the content configuration may include a recommendation to prepare for the type of medical procedure scheduled for the patient, a recovery process recommendation for the type of medical procedure scheduled for the patient, a nutritional recommendation, an exercise recommendation, a video image associated with a recommendation, a graphical image associated with a recommendation, a textual image associated with a recommendation, or a combination thereof. In some cases, the recorded medical information associated with the patient may include a medical condition associated with the patient, a demographic characteristic associated with the patient, a physiological measurement value, a patient generated response, a date of the type of medical procedure, or a combination thereof.

The message receiver 715 may receive an electronic inquiry message via an electronic messaging application from the patient.

The response component 720 may determine, automatically by the electronic messaging application, a response to the received electronic inquiry message based on content of the electronic inquiry message and recorded medical information associated with the patient. In some examples, the response component 720 may update the recorded medical information associated with the patient in a database of a patient management system based on determining the response.

In some examples, the response component 720 may transmit, to the patient, an electronic reminder message associated with the response to the received electronic inquiry message, a schedule of the type of medical procedure, or both. In some examples, the response component 720 may receive, from the patient, an electronic confirmation message indicating that the patient received the electronic reminder message, completed a task associated with the electronic reminder message, or both.

The configuration component 725 may update the content configuration for the patient portal based on the content of the electronic inquiry message and the response. In some examples, the configuration component 725 may display, via a graphical user interface, the content configuration for the patient portal, where the content configuration includes the determined patient care instructions.

The instructions component 730 may determine patient care instructions based on the content of the electronic inquiry message and the recorded medical information associated with the patient. In some examples, the instructions component 730 may transmit, to the patient, the determined patient care instructions.

The intervention action component 735 may determine an intervention action to respond to the electronic inquiry message based on the content of the electronic inquiry message and the recorded medical information associated with the patient. In some examples, the intervention action component 735 may determine a lack of response from the patient support system to the first request for the intervention action. In some examples, the intervention action component 735 may receive an indication of the intervention action for displaying in the content configuration, where the indication of the intervention action is received from the patient support system, the clinician, or both.

The request component 740 may transmit, to a clinician, a first request for the intervention action based on determining the intervention action. In some examples, the request component 740 may transmit, to a patient support system, a first request for the intervention action based on determining the intervention action. In some examples, the request component 740 may transmit, to a clinician, a second request for the intervention action based on determining the lack of response to the first request for the intervention action.

The patient management component 745 may transmit, to a patient management system, a patient communication history record associated with communication between the patient and the electronic messaging application. In some examples, the patient management component 745 may transmit, to a patient management system, an electronic status indicator associated with a status of the patient after a completion of the type of medical procedure scheduled for the patient.

FIG. 8 shows a diagram of a system 800 including a patient management server 805 that supports digital patient engagement in accordance with aspects of the present disclosure. The patient management server 805 may he an example of or include the components of patient management server 505, patient management server 605, or a patient management server as described herein. The patient management server 805 may include components for bi-directional voice and data communications including components for transmitting and receiving communications, including a communications manager 810, an I/O controller 815, a transceiver 820, an antenna 825, memory 830, and a processor 840. These components may be in electronic communication via one or more buses (e.g., bus 845).

The communications manager 810 may identify a content configuration for a patient portal based on a type of medical procedure scheduled for the patient, receive an electronic inquiry message via an electronic messaging application from the patient, determine, automatically by the electronic messaging application, a response to the received electronic inquiry message based on content of the electronic inquiry message and recorded medical information associated with the patient, and update the content configuration for the patient portal based on the content of the electronic inquiry message and the response.

The I/0 controller 815 may manage input and output signals for the patient management server 805. The I/O controller 815 may also manage peripherals not integrated into the patient management server 805. In some cases, the I/O controller 815 may represent a physical connection or port to an external peripheral. In some cases, the I/O controller 815 may utilize an operating system such as iOS®, ANDROID®, MS-DOS®, MS-WINDOWS®, OS/2®, UNIX®, LINUX®, or another known operating system. In other cases, the I/O controller 815 may represent or interact with a modem, a keyboard, a mouse, a touchscreen, or a similar device. In some cases, the I/O controller 815 may be implemented as part of a processor. In some cases, a user may interact with the patient management server 805 via the I/O controller 815 or via hardware components controlled by the I/O controller 815.

The transceiver 820 may communicate bi-directionally, via one or more antennas, wired, or wireless links as described above. For example, the transceiver 820 may represent a.

wireless transceiver and may communicate hi-directionally with another wireless transceiver. The transceiver 820 may also include a modem to modulate the packets and provide the modulated packets to the antennas for transmission, and to demodulate packets received from the antennas.

In some cases, the wireless device may include a single antenna 825. However, in some cases the device may have more than one antenna 825, which may be capable of concurrently transmitting or receiving multiple wireless transmissions.

The memory 830 may include RAM and ROM. The memory 830 may store computer-readable, computer-executable code 835 including instructions that, when executed, cause the processor to perform various functions described herein. In some cases, the memory 830 may contain, among other things, a BIOS which may control basic hardware or software operation such as the interaction with peripheral components or devices.

The processor 840 may include an intelligent hardware device, (e.g., a general-purpose processor a DSP, a CPU, a microcontroller, an ASIC, an FPGA, a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory (e.g., the memory 830) to cause the patient management server 805 to perform various functions (e.g., functions or tasks supporting digital patient engagement).

The code 835 may include instructions to implement aspects of the present disclosure, including instructions to support managing digital communications with a patient. The code 835 may be stored in a non-transitory computer-readable medium such as system memory or other type of memory. In some cases, the code 835 may not be directly executable by the processor 840 but may cause a computer (e.g., when compiled and executed) to perform functions described herein.

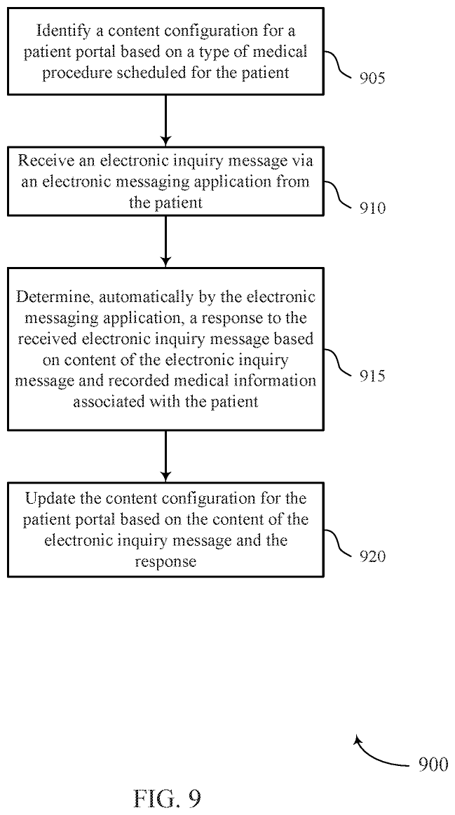

FIG. 9 shows a flowchart illustrating a method 900 that supports digital patient engagement in accordance with aspects of the present disclosure. The operations of method 900 may be implemented by a patient management server or its components as described herein. For example, the operations of method 900 may be performed by a communications manager as described with reference to FIGS. 5 through 8. In some examples, a patient management server may execute a set of instructions to control the functional elements of the patient management server to perform the functions described below. Additionally or alternatively, a patient management server may perform aspects of the functions described below using special-purpose hardware.

At 905, the patient management server may identify a content configuration for a patient portal based on a type of medical procedure scheduled for the patient. The operations of 905 may be performed according to the methods described herein. In some examples, aspects of the operations of 905 may be performed by a content identifier as described with reference to FIGS. 5 through 8.

At 910, the patient management server may receive an electronic inquiry message via an electronic messaging application from the patient. The operations of 910 may be performed according to the methods described herein. In some examples, aspects of the operations of 910 may be performed by a message receiver as described with reference to FIGS. 5 through 8.

At 915, the patient management server may determine, automatically by the electronic messaging application, a response to the received electronic inquiry message based on content of the electronic inquiry message and recorded medical information associated with the patient. The operations of 915 may be performed according to the methods described herein. In some examples, aspects of the operations of 915 may be performed by a response component as described with reference to FIGS. 5 through 8.

At 920, the patient management server may update the content configuration for the patient portal based on the content of the electronic inquiry message and the response. The operations of 920 may be performed according to the methods described herein. In some examples, aspects of the operations of 920 may he performed by a configuration component as described with reference to FIGS. 5 through 8.

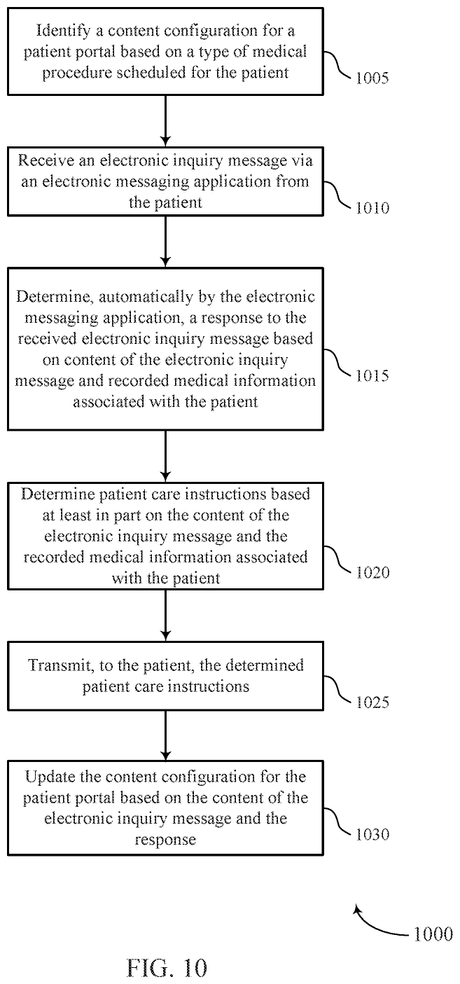

FIG. 10 shows a flowchart illustrating a method 1000 that supports digital patient engagement in accordance with aspects of the present disclosure. The operations of method 1000 may be implemented by a patient management server or its components as described herein. For example, the operations of method 1000 may be performed by a communications manager as described with reference to FIGS. 5 through 8. In some examples, a patient management server may execute a set of instructions to control the functional elements of the patient management server to perform the functions described below. Additionally or alternatively, a patient management server may perform aspects of the functions described below using special-purpose hardware.

At 1005, the patient management server may identify a content configuration for a patient portal based on a type of medical procedure scheduled for the patient. The operations of 1005 may be performed according to the methods described herein. In some examples, aspects of the operations of 1005 may be performed by a content identifier as described with reference to FIGS. 5 through 8.

At 1010, the patient management server may receive an electronic inquiry message via an electronic messaging application from the patient. The operations of 1010 may be performed according to the methods described herein. In some examples, aspects of the operations of 1010 may he performed by a message receiver as described with reference to FIGS. 5 through 8.

At 1015, the patient management server may determine, automatically by the electronic messaging application, a response to the received electronic inquiry message based on content of the electronic inquiry message and recorded medical information associated with the patient. The operations of 1015 may be performed according to the methods described herein. In some examples, aspects of the operations of 1015 may be performed by a response component as described with reference to FIGS. 5 through 8.

At 1020, the patient management server may determine patient care instructions based at least in part on the content of the electronic inquiry message and the recorded medical information associated with the patient. The operations of 1020 may be performed according to the methods described herein. In some examples, aspects of the operations of 1020 may be performed by an instruction component as described with reference to FIGS. 5 through 8.

At 1025, the patient management server may transmit, to the patient, the determined patient care instructions. The operations of 1025 may be performed according to the methods described herein. In some examples, aspects of the operations of 1025 may be performed by an instruction component as described with reference to FIGS. 5 through 8.

At 1030, the patient management server may update the content configuration for the patient portal based on the content of the electronic inquiry message and the response. The operations of 1030 may be performed according to the methods described herein. In some examples, aspects of the operations of 1030 may be performed by a configuration component as described with reference to FIGS. 5 through 8.

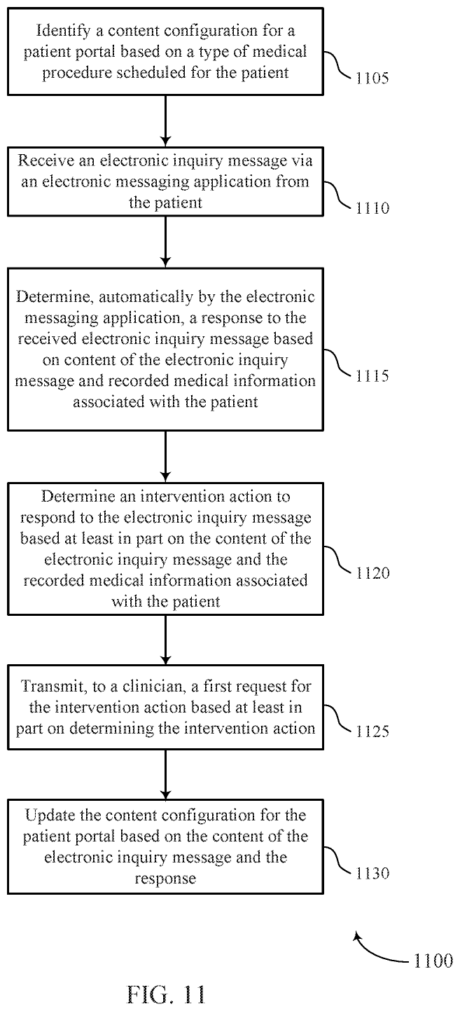

FIG. 11 shows a flowchart illustrating a method 1100 that supports digital patient engagement in accordance with aspects of the present disclosure. The operations of method 1100 may be implemented by a patient management server or its components as described herein. For example, the operations of method 1100 may be performed by a communications manager as described with reference to FIGS. 5 through 8. In some examples, a patient management server may execute a set of instructions to control the functional elements of the patient management server to perform the functions described below. Additionally or alternatively, a patient management server may perform aspects of the functions described below using special-purpose hardware.

At 1105, the patient management server may identify a content configuration for a patient portal based on a type of medical procedure scheduled for the patient. The operations of 1105 may be performed according to the methods described herein. In some examples, aspects of the operations of 1105 may be performed by a content identifier as described with reference to FIGS. 5 through 8.

At 1110, the patient management server may receive an electronic inquiry message via an electronic messaging application from the patient. The operations of 1110 may be performed according to the methods described herein. In some examples, aspects of the operations of 1110 may be performed by a message receiver as described with reference to FIGS. 5 through 8.

At 1115, the patient management server may determine, automatically by the electronic messaging application, a response to the received electronic inquiry message based on content of the electronic inquiry message and recorded medical information associated with the patient. The operations of 1115 may be performed according to the methods described herein. In some examples, aspects of the operations of 1115 may be performed by a response component as described with reference to FIGS. 5 through 8.

At 1120, the patient management server may determine an intervention action to respond to the electronic inquiry message based at least in part on the content of the electronic inquiry message and the recorded medical information associated with the patient. The operations of 1120 may be performed according to the methods described herein. In some examples, aspects of the operations of 1120 may be performed by an intervention action component as described with reference to FIGS. 5 through 8.

At 1125, the patient management server may transmit, to a clinician, a first request for the intervention action based at least in part on determining the intervention action. The operations of 1125 may be performed according to the methods described herein. In some examples, aspects of the operations of 1125 may be performed by an intervention action component as described with reference to FIGS. 5 through 8.

At 1130, the patient management server may update the content configuration for the patient portal based on the content of the electronic inquiry message and the response. The operations of 1130 may be performed according to the methods described herein. In some examples, aspects of the operations of 1130 may he performed by a configuration component as described with reference to FIGS. 5 through 8.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples,” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may he distinguished by following the reference label by a dash and a second label that distinguishes among the similar components. If just the first reference label is used in the specification, the description is applicable to any one of the similar components having the same first reference label irrespective of the second reference label.

Information and signals described herein may he represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may he implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same mariner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for managing digital communications with a patient, comprising:
identifying a content configuration for a patient portal based at east in part on a type of medical procedure scheduled for the patient;receiving an electronic inquiry message via an electronic messaging application from the patient;
determining, automatically by the electronic messaging application, a response to the received electronic inquiry message based at least in part on content of the electronic inquiry message and recorded medical information associated with the patient; and
updating the content configuration for the patient portal based at least in part on the content of the electronic inquiry message and the response.

2. The method of claim I, further comprising:
determining patient care instructions based at least in part on the content of the electronic inquiry message and the recorded medical information associated with the patient; and
transmitting, to the patient, the determined patient care instructions.

3. The method of claim 2, further comprising:
displaying, via a graphical user interface, the content configuration for the patient portal, wherein the content configuration comprises the determined patient care instructions.

4. The method of claim 1, further comprising:
determining an intervention action to respond to the electronic inquiry message based at least in part on the content of the electronic inquiry message and the recorded medical information associated with the patient; and
transmitting, to a clinician, a first request for the intervention action based at least in part on determining the intervention action.

5. The method of claim 1, further comprising:
determining an intervention action to respond to the electronic inquiry message based at least in part on the content of the electronic inquiry message and the recorded medical information associated with the patient; and
transmitting, to a patient support system, a first request for the intervention action based at least in part on determining the intervention action.

6. The method of claim 5, further comprising:
determining a lack of response from the patient support system to the first request for the intervention action; and
transmitting, to a clinician, a second request for the intervention action based at least in part n determining the lack of response to the first request for the intervention action.

7. The method of claim 6, further comprising:
receiving an indication of the intervention action for displaying in the content configuration, wherein the indication of the intervention action is received from the patient support system, the clinician, or both.

8. The method of claim 1, further comprising:
updating the recorded medical information associated with the patient in a database of a patient management system based at least in part on determining the response.

9. The method of claim 1, further comprising:
transmitting, to a patient management system, a patient communication history record associated with communication between the patient and the electronic messaging application.

10. The method of claim 1, further comprising:
transmitting, to a patient management system, an electronic status indicator associated with a status of the patient after a completion of the type of medical procedure scheduled for the patient.

11. The method of claim 1, further comprising:
transmitting, to the patient, an electronic reminder message associated with the response to the received electronic inquiry message, a schedule of the type of medical procedure, or both; and
receiving, from the patient, an electronic confirmation message indicating that the patient received the electronic reminder message, completed a task associated with the electronic reminder message, or both.

12. The method of claim 1, wherein the content configuration comprises a recommendation to prepare for the type of medical procedure scheduled for the patient, a recovery process recommendation for the type of medical procedure scheduled for the patient, a nutritional recommendation, an exercise recommendation, a video image associated with a recommendation, a graphical image associated with a recommendation, a textual image associated with a recommendation, or a combination thereof.

13. The method of claim 1, wherein the recorded medical information associated with the patient comprises a medical condition associated with the patient, a demographic characteristic associated with the patient, a physiological measurement value, a patient generated response, a date of the type of medical procedure, or a combination thereof.

14. An apparatus for managing digital communications with a patient, comprising:
a processor;
memory in electronic communication with the processor; and
instructions stored in the memory and executable by the processor to cause the apparatus to:
identify a content configuration for a patient portal based at least in part on a type of medical procedure scheduled for the patient;
receive an electronic inquiry message via an electronic messaging application from the patient;
determine, automatically by the electronic messaging application, a response to the received electronic inquiry message based at least in part on content of the electronic inquiry message and recorded medical information associated with the patient; and
update the content configuration for the patient portal based at least in part on the content of the electronic inquiry message and the response.

15. The apparatus of claim 14, wherein the instructions are further executable by the processor to cause the apparatus to:
determine patient care instructions based at least in part on the content of the electronic inquiry message and the recorded medical information associated with the patient; and
transmit, to the patient, the determined patient care instructions.

16. The apparatus of claim 15, wherein the instructions are further executable by the processor to cause the apparatus to:
display, via a graphical user interface, the content configuration for the patient portal, wherein the content configuration comprises the determined patient care instructions.

17. The apparatus of claim 14, wherein the instructions are further executable by the processor to cause the apparatus to:
determine an intervention action to respond to the electronic inquiry message based at least in part on the content of the electronic inquiry message and the recorded medical information associated with the patient; and
transmit, to a clinician, a first request for the intervention action based at least in part n determining the intervention action.

18. The apparatus of claim 14, The apparatus of claim 14, wherein the instructions are further executable by the processor to cause the apparatus to:
determine an intervention action to respond to the electronic inquiry message based at least in part on the content of the electronic inquiry message and the recorded medical information associated with the patient; and
transmit, to a patient support system, a first request for the intervention action based at east in part on determining the intervention action.

19. The apparatus of claim 18, The apparatus of claim 14, wherein the instructions are further executable by the processor to cause the apparatus to:
determine a lack of response from the patient support system to the first request for the intervention action; and
transmit, to a clinician, a second request for the intervention action based at least in part on determining the lack of response to the first request for the intervention action.

20. A non-transitory computer readable medium storing code for managing digital communications with a patient, the code comprising instructions executable by a processor to:
identify a content configuration for a patient portal based at least in part on a type of medical procedure scheduled for the patient;
receive an electronic inquiry message via an electronic messaging application from the patient;
determine, automatically by the electronic messaging application, a response to the received electronic inquiry message based at least in part on content of the electronic inquiry message and recorded medical information associated with the patient; and
update the content configuration for the patient portal based at least in part on the content of the electronic inquiry message and the response.
